(19) United States
(12) Patent Application Publication
(10) Pub. No.: US 2017/0306835 A1
(43) Pub. Date: Oct. 26, 2017

(54) Method and system for generating a mechanical output and producing reaction products in a parallel manner

(71) Applicant: Linde Aktiengesellschaft, München (DE)

(72) Inventor: David Bruder, München (DE)

(73) Assignee: Linde Aktiegesellschaft, Munchen (DE)

(21) Appl. No.: 15/508,575

(22) PCT Filed: Sep. 2, 2015
(86) PCT No.: PCT/EP2015/070090

§ 371 Date: Mar. 3, 2017

(30) Foreign Application Priority Data
Sep. 5, 2014 (EP) 14183742.7

(51) Int. Cl. F02B 43/12; B01J 19/24; C07C 5/327; F02C 7/08; B01J 19/00; F01K 13/00; C07C 4/04; F02B 43/10

(52) U.S. Cl. CPC F02B 43/12; F01K 13/006; B01J 19/2415; C07C 4/04; C07C 5/327; F02C 7/08; B01J 19/0013; F02B 2043/103; B01J 2219/00117; F05D 2220/32; F05D 2220/76

(57) ABSTRACT

A process for the combined generation of mechanical power and manufacture of hydrocarbons is proposed, wherein in order to generate the mechanical power at least one internal combustion engine (1) is fired up, thereby producing a combustion exhaust gas (c), and in order to produce the hydrocarbons at least one reactor (2) is heated using a fuel (e) and a combustion support gas (d). The invention provides that at least a proportion of the combustion support gas (d) is heated by indirect heat exchange with at least a proportion of the combustion exhaust gas (c) from the internal combustion engine (1). The present invention also relates to a corresponding installation (100, 200).

The invention relates to a process and an installation for the parallel generation of mechanical power and manufacture of hydrocarbons according to the pre-characterising clauses of the independent claims.

### PRIOR ART

In a number of processes for producing chemical reaction products, reactors are used which comprise reactor tubes heated by burners, through which a feed is passed and is then at least partly reacted to form the desired reaction products. Examples of processes of this kind are steam cracking, the dehydrogenation of alkanes and also the production of synthesis gas or ammonia.

Corresponding methods and apparatus are extensively described in the literature. For methods and apparatus for steam cracking, reference may be made for example to the article “Ethylene” in Ullmann's Encyclopedia of Industrial Chemistry, online since 15 Apr. 2007, DOI 10.1002/14356007.a10_045.pub2. Methods and apparatus for the dehydrogenation of alkanes, particularly of propane to propylene and isobutane to isobutene, can be found for example in the article “Propene” in Ullmann's Encyclopedia of Industrial Chemistry, Online edition, 15 Jun. 2000, DOI 10.1002/14356007.a22_211, section 4.3., “Propane Dehydrogenation”.

It has long been desired to couple such reactors with apparatus for generating mechanical power. The latter may be realized for example using internal combustion engines, particularly gas turbines. Known gas and steam processes and corresponding apparatus serve as an example of combined processes of this kind.

In a gas and steam process as illustrated in FIG. 1 and described hereinafter, an oxygen-containing combustion support gas, typically air, is aspirated by means of a gas turbine and compressed. A suitable fuel, typically natural gas or some other gas mixture, is introduced into a combustion chamber of the gas turbine and burned under pressure in the atmosphere formed by the combustion support gas. Decompression of on the combustion exhaust gas thus formed (also known as hot gas) drives an expansion stage of the gas turbine and through this a generator coupled to the gas turbine.

Heat still present in the combustion exhaust gas downstream of the gas turbine can be used in a waste heat steam generator (so-called Heat Recovery Steam Generator, HRSG) to produce pressurised steam. The pressurised steam can be used to drive a steam turbine. The power of the steam turbine is typically used to further generate electrical energy, in the generator coupled to the gas turbine or in another generator.

Combined processes in which gas turbines and the heated reactors described herein before are used are fundamentally also known, as explained with reference to FIG. 3. As described in detail hereinafter, however, there is a significant reduction in the radiation zone efficiency of the reactor used in equipment of this kind. The overall efficiency of such equipment is therefore at best slightly above that of separate apparatus for generating electrical energy and for recovering hydrocarbons. The low efficiency advantage of combined apparatus therefore does not normally justify the expense of coupling them.

Particularly in apparatus of this kind there is a dependency in the operation of the heated reactor on the operation of the gas turbine. If the latter goes out of commission, in extreme cases the reactor also has to be shut down, leading to correspondingly costly loss of production. Typically, the above-mentioned reactors may be designed for long-term operation over a number of years or are constructed in the form of a number of parallel units which are maintained or regenerated alternately. In steam cracking processes, for example, five to ten reactors may be in operation at all times, one being in so-called de-coking mode. However, a gas turbine requires significantly more frequent maintenance.

GB 2148734 A discloses a power plant with a high-speed fluidised bed reactor. A subdivided heat transfer fluidised bed is provided which is designated to swirl hot ash generated by the fluidised bed reactor and to extract heat from it. Means are provided which are designated to control those parts of the hot ash which are circulated through the sections of the heat transfer bed that are formed by the subdivisions, in order to control the power gains of the sections, respectively. A section of the subdivided fluidised bed can generate process steam and the other section can provide hot process air for a turbine.

From U.S. Pat. No. 5,048,284 A and from GB 2 296 719 A combined methods are known in which an autothermal reforming is performed and a turbine is operated. From FR 1 445 870 A a reforming reactor is known which is operated in combination with a turbine.

The problem of the present invention is therefore to improve combined methods for generating electrical energy and for manufacturing hydrocarbons, particularly in terms of their efficiency.

### DISCLOSURE OF THE INVENTION

This problem is solved by a method and an apparatus for generating mechanical power and for manufacturing hydrocarbons having the features of the independent claims. Preferred embodiments are the subject of the dependent claims and the description that follows.

Before the features of the present invention are explained, their basis and the terminology used will be explained.

In the following description, reference is frequently made to the efficiencies of thermal processes; the following definitions apply:

The technical combustion (“thermal”) efficiency (FTW, ny_FTW) denotes the proportion of heating power introduced (P_supplied) which is not lost to the environment through the combustion exhaust gas (P_exhaust gas):

ny_FTW=1−P_exhaust gas/P_supplied

The losses to the environment caused by the thermal conduction of hot components are not taken into account here as they are typically significantly less than the exhaust gas losses.

The radiation zone efficiency (SZW, ny_SZ) denotes the proportion of the heating power introduced (P_supplied) which is transferred indirectly to a process medium in a firing chamber (P_process):

ny_SZ=P_process/P_supplied

The transfer typically takes place at temperatures significantly above 1,000° C. and preferably by radiation. Typical radiation zone efficiencies of reactors heated exclusively directly, i.e. only by means of burners but not, for example, by means of pre-heated combustion air, for steam cracking amount to approximately 0.42 (42%).

The electrical efficiency (ny_el) denotes the proportion of the heating power introduced (P_supplied) of a heat power process which is released as net power in the form of electrical power (the net power denotes the power of the thermal power process minus the power required for subsidiary equipment such as pumps and compressors):

ny_el=P_el/P_supplied

The term “energy efficiency” is used here generally as a comparative term which evaluates or quantifies the heating power required by different processes or combined processes in order to produce a specific quantity of one or more products or to generate a specific amount of electrical power. The term is used, for example, for the generation of electrical power by means of a one-stage steam process, a gas turbine and a combined gas and steam process. Typically the efficiency increases in the order specified, i.e. the heating power used for a specific amount of electric current generated falls.

The firing power of a fuel is typically related to the lower heating or calorific value (Hu) within the scope of this application. It refers to the maximum amount of heat that can be used in a combustion in which there is no condensation of the water vapour contained in the exhaust gas, based on the amount of fuel used.

In common parlance, the term “gas turbine” refers to an arrangement which, as already mentioned, comprises a compression stage, the expansion stage as the actual gas turbine and a combustion chamber connected between the compression stage and the expansion stage. The combustion chamber is supplied through the compression stage with a compressed combustion support gas such as air. Through a fuel inlet the fuel (which is generally liquid or gaseous) enters the combustion chamber. The fuel is burned with the gas mixture in the combustion chamber, to form a combustion exhaust gas, the so-called hot gas.

The hot gas is decompressed in the expansion stage, at which point thermal power is converted into mechanical power. The mechanical power is taken off by means of one or more shafts. Some of the mechanical power is used to operate the compression stage, while the remainder is used, for example, to drive a generator. After the decompression the combustion gas is expelled as exhaust gas or, as in the present case, used as a heating medium.

Within the scope of this application the term “combustion support gas” is used to convey the idea that combustion of a fuel does not necessarily have to take place with air (“combustion air”) but can also take place in a different gas mixture, although it must contain oxygen:

As in the case of gas turbines, fired reactors as mentioned hereinbefore are supplied with a combustion support gas in addition to the fuel which is burned in corresponding burners for underfiring. Typically, air is used as the combustion support gas. However, it is also possible for the combustion exhaust gas from a gas turbine to be used at least partly as the combustion support gas. This is possible because the combustion of a fuel in a gas turbine typically takes place with a significantly hyperstoichiometric oxygen supply. Therefore there is still a considerable amount of oxygen present in the combustion exhaust gas, enabling the combustion exhaust gas to be used as a combustion support gas in the reactor. In addition to a combustion exhaust gas from a gas turbine, additional air or an oxygen-containing gas mixture may also be used in such a reactor for regulating the combustion. The problems that arise when using a combustion exhaust gas from a gas turbine as the combustion support gas are described below and form the starting point for the present invention.

### Advantages of the Invention

The present invention starts from a fundamentally known method for the combined generation of mechanical power and manufacture of hydrocarbons, in which, in order to generate the mechanical power, at least one internal combustion engine is fired up, producing a combustion exhaust gas, and wherein, in order to produce the hydrocarbons, at least one reactor is heated using a fuel and a combustion support g as.

According to the invention, it is provided that at least a proportion of the combustion support gas is heated by indirect heat exchange with at least a proportion of the combustion exhaust gas from the internal combustion engine. In other words, according to the present invention, not all the combustion exhaust gas is supplied to the reactor, but only a proportion of it. An externally supplied combustion support gas, for example air, is preheated by means of another part or all of the combustion exhaust gas.

Where the present application speaks of “supplying” or “feeding” a fuel, a combustion support gas and/or a combustion exhaust gas into a reactor, this means feeding it into corresponding burners or a combustion chamber, not into the reaction zone, for example the reaction tubes of a reactor. A gas mixture passed through the reaction zone, for example, the reaction tubes, is referred to as a (tube-side) process gas within the scope of this application.

The present invention is particularly suitable for processes in which the mechanical power generated is used at least partly to drive a generator, i.e. is converted at least partly into electrical power. However, the invention may also be used to advantage when at least one shaft, for example, of a compressor and/or a pump, is driven at least partly by means of the mechanical power. In this case, the driven unit may, for example, be part of the process used to produce the hydrocarbons. For example, the mechanical power may be used to drive a compressor for compressing a process gas or steam.

The present invention is based on the finding that the efficiency, more precisely the radiation zone efficiency defined hereinbefore, of the reactor in corresponding combined processes according to the prior art is significantly reduced inter alia, by the fact that, in terms of energy balance, a combustion exhaust gas from a gas turbine, fed directly into the reactor to support the combustion of the fuel, has already had a significant proportion of energy removed in the gas turbine. In particular, this can be illustrated by means of the oxygen content of a corresponding combustion exhaust gas:

There is necessarily a reduction in the oxygen content of the combustion support gas in the gas turbine, even if combustion takes place therein with a significantly hyperstoichiometric oxygen supply. If air with a natural oxygen content of about 21% is used as the combustion support gas in the gas turbine, as is usual, this oxygen content reduces to about 14% in the combustion exhaust gas.

In corresponding reactors, particularly reactors used for steam cracking, the radiation zone efficiency essentially depends, however, on the temperature which can be achieved by burning the fuel and which can be transmitted to the feed passed through the reaction tubes. In conventional processes, i.e. self-sustaining reactors which are not coupled to gas turbines, an adiabatic combustion temperature of about 2,000° C. is reached when air is used as the combustion support gas. The adiabatic combustion temperature is the temperature that would be obtained after completion of combustion if the gas mixture did not exchange any heat with the environment during the combustion. This is therefore a theoretical temperature which is not actually achieved since such a reactor does not operate adiabatically in reality. The adiabatic combustion temperature is, however, a comparative term used in the art, which most conveniently describes the variable on which the radiation zone efficiency depends.

If a combustion support gas contains less oxygen because the latter has been partly reacted in an upstream gas turbine, only adiabatic temperatures of about 1750° C. can be achieved. Although the combustion exhaust gas leaves the gas turbine at about 600° C., for example, and therefore a considerable amount of heat is additionally available, the reduced oxygen content is still no longer sufficient to reach the adiabatic combustion temperature of conventional reactors.

If it is assumed, in simple terms (again from the point of view of energy balance), that the gas turbine discharges about one third of the heat power supplied to it as shaft power and the heat power supplied to the gas turbine makes up about one third of the heat power supplied to the gas turbine and the reactor as a whole, one ninth of the total heat power is removed from the combustion exhaust gas in the form of the shaft power. Accordingly, the adiabatic combustion temperature is therefore reduced by about one ninth.

As already explained, the present invention therefore proposes that not all the combustion exhaust gas should be fed into the reactor and used to support the combustion of the fuel, but at most a proportion thereof. Thus, in contrast to conventional coupled installations, partially or exclusively external combustion support gas which is not formed from the combustion exhaust gas, e.g. fresh combustion air, is supplied to the reactor. The actual coupling of the gas turbine with a corresponding reactor is carried out by means of a preheating device which comprises, for example, one or more suitable heat exchangers for indirect heat exchange. As a result of the indirect heat exchange of the combustion exhaust gas with the combustion support gas, the temperature thereof is indeed utilised (i.e. heat power is transferred), but the oxygen content of the combustion support gas is not affected. In this way, for example, fresh air containing about 21% oxygen can be heated as the combustion support gas and fed into the reactor. As a result, once again the adiabatic combustion temperatures of about 2,000° C. described previously (when some of the combustion exhaust gas is used only for preheating and some is fed into the reactor) or even higher (when used exclusively for preheating) can be achieved as described hereinafter.

The above-mentioned partial use of the combustion exhaust gas only for preheating on the one hand and the partial feeding into the reactor, on the other hand, comprises, for example, mixing part of the combustion exhaust gas with “fresh” combustion support gas, e.g. air, and thereby achieving a defined oxygen content. For example, an oxygen content of about 19% can be selected. Another part of the combustion exhaust gas is not fed into the reactor but used only to preheat the combustion support gas by indirect heat exchange. With an assumed temperature of the combustion exhaust gas of about 600° C. the above-mentioned adiabatic combustion temperature of about 2,000° C. can be achieved in the reactor and hence a radiation zone efficiency can be achieved comparable to that obtained in conventional reactors. Therefore the operation of the reactor has to be adjusted only slightly, if at all.

Another essential advantage of the present invention is that, even when the gas turbine is out of commission or in need of maintenance, the reactor can continue to be operated. In this case, air which has not been preheated can be used as a combustion support gas, for example. Alternatively, in this case, it is also possible to preheat the combustion support gas in some other way, for example, using steam and/or flue gas. Corresponding preheating equipment therefore has only to be designed for short term operation and is correspondingly inexpensive.

It should be mentioned at this point that, in conventional apparatus such as that illustrated in FIG. 3, for example, additional combustion support gas can be fed in, in addition to the combustion exhaust gas from the gas turbine, all of which is fed into the reactor. However, this is conventionally done only in order to achieve a regulating variable for increasing the independence between the gas turbine and the reactor. The problem of the reduced oxygen content and the impaired radiation zone efficiency in the reactor inherently cannot be solved by this method.

Within the scope of the present invention, by contrast, the radiation zone efficiency of the reactor can be significantly increased. The reactor can be operated at the radiation zone efficiency level which can also be achieved in a self-sustaining reactor, as explained hereinbefore. However, it is also possible to increase the radiation zone efficiency further by preheating and by achieving even higher temperatures in the reactor.

The embodiments of the present invention recited in the claims and explained in part hereinbefore will now be summarised:

In particular, the method of the present invention is suitable for use in the tube reactors mentioned hereinbefore, i.e. in apparatus in which the at least one reactor is embodied as a tube reactor in which, in a radiation zone, reaction tubes are heated from the outside by burners in which the fuel is burned. Conventional reactors operated in self-sustaining manner comprise feed openings through which the combustion support gas is fed in. Inside the reactor or the combustion chamber of a corresponding reactor there is a slight negative pressure which is produced by a blower in the flue gas channel. A combustion support gas is therefore automatically aspirated. By contrast, the present invention may comprise feeding a combustion support gas into a corresponding reactor or its combustion chamber under a slight positive pressure by means of a blower. Such a feed method is typical, for example, for preheating air in hydrogen reforming processes.

The method according to the present invention is particularly suitable for the steam cracking processes mentioned hereinbefore, i.e. for processes in which, in order to produce the (olefinic) hydrocarbons, a feed containing hydrocarbons is fed with steam through the reaction tubes of the reactor configured as a tube reactor. In corresponding steam cracking processes, the above-mentioned temperatures prevail in the radiation zone. However, the invention is similarly also suitable for catalytic processes, for example the previously mentioned processes for alkane dehydrogenation, i.e. for processes comprising reactors in which a catalyst is provided in the reaction tubes, or hydrogen reforming processes.

As already mentioned, the process according to the invention is particularly advantageous because the temperatures that can be achieved thereby make it possible to reach a high radiation zone efficiency for the reactor. This means, in other words, that the method is used in cases where at least a region of the at least one reactor is heated to an adiabatic combustion temperature of typically 1,500-2,500° C. by heating, using the fuel and the combustion support gas.

Suitable internal combustion engines for use in the present invention are, in particular, gas turbines, as they have a high nominal output at relatively low cost whilst having good mechanical or electrical efficiency. Gas turbines are therefore typically used in power stations. The mechanical efficiency of a gas turbine alone is also typically no higher than that of a correspondingly configured diesel engine or a coal and steam power station. As the temperature of the combustion exhaust gas is about 600° C. in a diesel engine and about 700-1,000° C. in a petrol engine, internal combustion engines of this kind are also suitable for use in the present invention.

Whereas conventionally one disadvantage of the use of a gas turbine is the relatively high quality fuel (gas) used, this is actually an advantage in the present invention: in the methods of producing reaction products which are under discussion here (for example in steam cracking processes and hydrogen reforming) a so-called tail gas which is of high value from the point of view of combustion is obtained as a residual gas. This is a methane-containing fraction or a mixture of carbon monoxide, carbon dioxide and hydrogen (synthesis gas). The (partial) process carried out within the scope of the invention for producing the reaction products thus yields a suitable fuel for a gas turbine. Obviously, a corresponding gas mixture may also be combusted in an engine. This brings about a further synergistic integration of corresponding parts of the equipment.

It is particularly advantageous if the exhaust gas from the internal combustion engine is provided at a temperature level of less than 650° C. as in this case a combustion support gas can be heated particularly effectively and cheaply. The material costs, for example, for the heat exchangers used, are still markedly low at such temperatures. However, in general, the exhaust gas from the internal combustion engine can be provided at a temperature level of 500-1,000° C., particularly at a temperature level of 600-700° C. or at a temperature level of 500-650° C.

In a process according to one embodiment of the invention, advantageously some of the exhaust gas from the internal combustion engine is used to heat the combustion support gas by indirect heat exchange and some of the exhaust gas from the internal combustion engine is combined with the combustion support gas and supplied together with it to the at least one reactor. This use of the exhaust gas partially for preheating and partially for feeding into the reactor allows a particularly favourable combination of a gas turbine or another internal combustion engine and a reactor. In this case, the conditions in the reactor can be approximated to those of conventional self-sustaining reactors, which means that no or only minor changes are needed to the mode of operation of corresponding reactors and/or their constructive configuration. The reaction tubes and the apparatus for utilising waste heat (in the so-called convection zone) can be retained.

In new installations, however, it may prove advantageous to use the exhaust gas from the internal combustion engine completely to heat the combustion support gas by indirect heat exchange and not to supply it to the at least one reactor. The at least one reactor therefore receives the total oxygen content of the combustion support gas, for example air, in addition to the heat of the exhaust gas, so that the temperatures in such a reactor can be increased further. In this way the radiation zone efficiency of a corresponding reactor is increased considerably. The fuel consumption at the reactor can be reduced accordingly by this method.

The present invention in particularly suitable for use with natural gas, a methane-containing gas mixture and/or synthesis gas as fuel and/or air as the combustion support gas. As already mentioned, corresponding fuels may also be typical residual gases from corresponding processes for manufacturing reaction products (for example from processes for steam cracking, synthesis gas production or hydrogen reforming). The present invention makes it possible, in particular, to save fuel by increased efficiency.

A further advantage of the method according to the invention is obtained if pressurised steam is produced from the waste heat from the at least one reactor and is used to drive at least one shaft, particularly a shaft of a generator. In this way, further mechanical power can be obtained and used profitably, even if the corresponding pressurised steam is at a lower pressure. The steam obtained by means of the waste heat from the at least one reactor is basically only a by-product by means of which the heat which cannot be used for the reaction (waste heat) can be profitably used. In a theoretical ideal case, only reaction heat would be produced in the reactor and no waste heat, i.e. no steam.

The advantage of the present invention is that the amount of pressurised steam produced by means of the waste heat from the reactor or reactors can be minimised. The pressurised steam produced by means of the waste heat from the reactor or reactors is obtained with significantly higher energy losses than in the power station steam process (a multi-stage process which is more efficient at its peak at higher pressures/temperatures). Pressurised steam can, for example, be used with almost 100% efficiency in the steam cracking processes partly as a heating steam for preheating the feed stream or streams. In the case of the generation of mechanical power in a turbine, the efficiency is worse by approximately a factor of 2 than in a steam power process.

For the features and advantages of the apparatus provided according to the invention for the generation of mechanical power and for the manufacture of hydrocarbons, which is arranged particularly for carrying out a process as explained hereinbefore, reference is specifically made to the foregoing remarks.

The invention and specific embodiments of the invention are illustrated in the appended drawings by comparison with the prior art.

### BRIEF DESCRIPTION OF THE DRAWINGS

FIG. 1 shows a simplified schematic representation of a gas and steam power station according to the prior art.

FIG. 2 shows a simplified schematic representation of a fired reactor operated according to the prior art.

FIG. 3 shows a simplified schematic representation of an apparatus with a gas turbine and a fired reactor according to the prior art.

FIG. 4 shows a simplified schematic representation of an apparatus with a gas turbine and a fired reactor according to one embodiment of the invention.

FIG. 5 shows a simplified schematic representation of an apparatus with a gas turbine and a fired reactor according to one embodiment of the invention.

In the Figures, corresponding elements have been given identical reference numerals and are not described repeatedly, in the interest of clarity. The same is also true of the fluid streams shown which are indicated by lower case letters, even if they are provided in different amounts, as explained hereinafter.

In all the embodiments illustrated, a reactor, if shown, is arranged to carry out a steam cracking process, i.e. it is supplied with a hydrocarbon-containing feed stream which is mixed with steam. The fuel used is a suitable combustion gas as described above, while air is used as the combustion support gas. However, the equipment shown is theoretically also suitable for carrying out other processes for manufacturing reaction products or using other fuels and combustion support gases. Although the following description frequently refers to “a” reactor or “a” gas turbine, corresponding installations may also comprise a number of reactors or gas turbines.

### DETAILED DESCRIPTION OF THE DRAWINGS

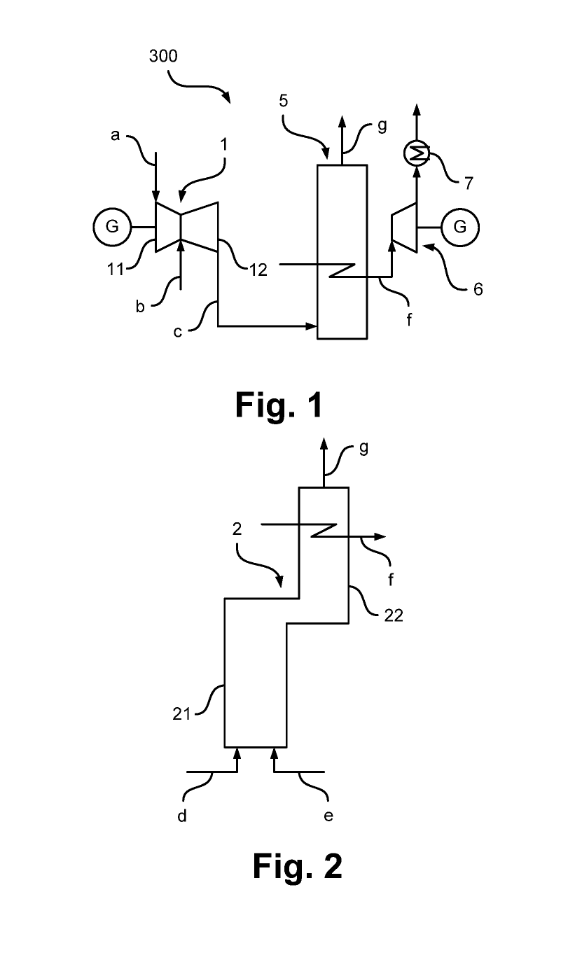

FIG. 1 shows a simplified schematic representation of a gas and steam power station according to the prior art, which is generally designated 300.

The gas and steam power station 300 comprises as central components a gas turbine 1 which, as described hereinbefore, comprises a compression stage 11 and an expansion stage 12 as well as a combustion chamber arranged between the compression stage 11 and the expansion stage 12 but not separately shown here. A generator G is driven by the gas turbine 1. The gas turbine 1 is supplied with a combustion support gas a which is compressed in the compression stage 11. A fuel b is fed into the combustion chamber (not shown) of the gas turbine 1 and is burned under pressure in the combustion chamber in an atmosphere created by the combustion support gas a.

Typically, the combustion takes place with a significantly hyperstoichiometric oxygen supply, for example at a lambda value of about 3, so that a combustion exhaust gas c (hot gas) formed during the combustion and allowed to expand in the expansion stage 12 of the gas turbine still has a considerable oxygen content. If air with a natural oxygen content of about 21% is used as the combustion support gas a, the combustion exhaust gas c still has an oxygen content of about 14%.

The combustion exhaust gas c, which may be at a temperature of 600° C., for example, is supplied to a heat recovery steam generator 5 in the gas and steam power station 300. Typically, little additional fuel is supplied to the heat recovery steam generator 5, i.e. the heat recovery steam generator 5 mainly uses the sensible heat of the combustion exhaust gas c is used in. A correspondingly cooled combustion exhaust gas g is discharged from the heat recovery steam generator 5.

In the highly simplified representation of FIG. 1, pressurised steam f is produced. Typically, in corresponding gas and steam power stations 300, pressurised steam f is generated at three pressure levels. The pressure levels are, for example, approximately 130, 30 and 8 bar, steam being partly removed from a turbine at an intermediate pressure (by “tapping”) at the middle and low pressure levels, and steam at the middle pressure level being heated to about 570° C., starting from the saturated steam temperature (“intermediate superheating”). The purpose of this procedure is to minimise energy losses by transferring the heat from the combustion exhaust gas c to feedwater or steam with the smallest possible temperature difference.

The pressurised steam f is used in a decompression turbine 6 (steam turbine) to produce shaft power (mechanical power). This power is in turn converted by a generator G into electrical power. This generator may be the same as the generator G coupled to the gas turbine 1 or it may be provided separately. The decompressed stream of vapour (not designated in FIG. 1) is cooled in a cooler 7, for example using cooling water. The steam condensate obtained is recycled into the process (using a so-called boiler feed water pump).

Typical characteristic values of a gas and steam power station 300 are illustrated below. The corresponding variables are given for a net electric power of 100 MW, as this is the order of magnitude required in an installation for steam cracking of a size corresponding to the prior art. In the power station field, net powers of 80-400 MW per gas turbine unit are typical. Approximately 619,000 standard cubic metres per hour (Nm³/h) of combustion air are used as combustion support gas a as well as an underfiring power in the form of the fuel b of about 180 MW. Corresponding values are summarized in the table hereinafter. Any rounding-up errors have been disregarded.

In the case illustrated, typically about 640,000 Nm³/h of the combustion exhaust gas c are formed. The amount of cooled combustion exhaust gas g in this case is also about 640,000 Nm³/h if there is no additional firing up in the heat recovery steam generator 5.

Typically, the electrical efficiency of the gas turbine 1 and of the generator connected thereto is about 0.36 (36%). The electrical efficiency of the decompression turbine 6, based on the energy supplied to the heat recovery steam generator 5, is about 0.32 or, based on the total energy used, about 0.20. The proportion of the total electrical power of a corresponding gas and steam power station made up by the decompression turbine 6 is also about 0.36, for example, in the embodiment shown. The thermal efficiency without taking the cooler 7 into consideration is about 0.82 in the embodiment. (The thermal efficiency depends on the condensation temperature, the quantity of steam, etc., and varies within the range from about 0.75 to about 0.85.) The thermal efficiency is often not particularly meaningful in the present case, as, even if the majority of the heat is taken from the flue gas, the mere provision of hot water or steam, for example, scarcely increases the efficiency of corresponding installations, unless it can be used with a high efficiency.

Of the roughly 180 MW underfiring power of the fuel b, against this background, in the generator G coupled to the gas turbine 1, roughly 64 MW are obtained as electrical power, about 112 MW are transferred as sensible heat into the combustion exhaust gas c and thermal losses typically correspond to about 3 MW. In turn, of the roughly 112 MW of perceptible heat in the combustion exhaust gas c, typically about 3 MW remain in the cooled combustion exhaust gas g, the cooled combustion exhaust gas g being cooled to a temperature of about 128° C. Of the remaining approximately 80 MW, 36 MW are obtained as electrical power in the generator G coupled to the decompression turbine 6 and roughly 44 MW are discharged in the cooler 7.

With a total heating power of about 180 MW and an electrical power of about 100 MW for the two generators G, the total electrical efficiency of a gas and steam power station 300 is about 0.56 in the embodiment. In a test run, efficiencies up to a maximum 0.61 were achieved in large gas and steam power stations (with approximately 800 MW power) but these drop significantly when the cooling water is warmer.

FIG. 2 shows a fired reactor according to the prior art in a simplified schematic representation, the reactor being generally designated 2. As explained previously, fired reactors of this kind may typically be used to produce hydrocarbons or synthesis gas, for example by steam cracking. A corresponding reactor 2, as is generally known, typically comprises a radiation zone 21 and a convection zone 22. In the radiation zone 21, typically a number of burners are arranged (not shown) which are supplied with a fuel d. The combustion is made possible by the supply of a combustion support gas e. In the radiation zone 21 and convection zone 22 there are typically reaction tubes which are heated from the outside by corresponding burners.

In a fired reactor 2, as well, waste heat is used for the most part for producing pressurised steam f but the latter is typically comparatively unsuitable to be used with a satisfactory degree of efficiency for the generation of electrical energy. The poorer utility value of the pressurised steam f from a typical fired reactor 2 as shown in FIG. 2 results from its comparatively low temperature and its comparatively low pressure and the fact that only one steam level is realized (and therefore the comparatively large energy loss during steam generation). Whereas in typical gas and steam power stations, for example a gas and steam power station 300 as shown in FIG. 1, pressurised steam f is obtained at 130 bar and 570° C., the pressure of the pressurised steam f from the fired reactor 2 as shown in FIG. 2 is typically only 120 bar and its temperature is typically only 520° C. Typically, corresponding pressurised steam f from fired reactors 2 is used to recover shaft power (e.g. in a steam cracking apparatus) and used as heating steam. Here, too, a cooled combustion exhaust gas g is obtained.

Assuming, in a corresponding consideration of the energy balance, an underfiring power of approximately 1,000 MW (typically distributed over a number of reactors) in the form of the fuel d and assuming a supply of, for example, about 1,067,000 Nm3/h of combustion air as the combustion support gas e, with a typical radiation zone efficiency of about 0.42 (which is a typical value for a reactor used for a steam cracking process) in the radiation zone 21, about 512 MW or about 595 t/h of pressurised steam f can be obtained from the waste heat from the reactor 2. Approximately 60 MW go into the cooled flue gas g, which is removed in a quantity of about 1,172,000 Nm3/h and at a temperature of about 128° C. The “missing” heating power of 428 MW is discharged in the form of chemical bonding energy and sensible heat in the tube-side process gas, i.e. not in the flue gas stream but from the reaction zone of the reactor 2. This value is the same for all the reactors 2 in the following Figures provided by way of example here, as the same amount of reaction product is produced.

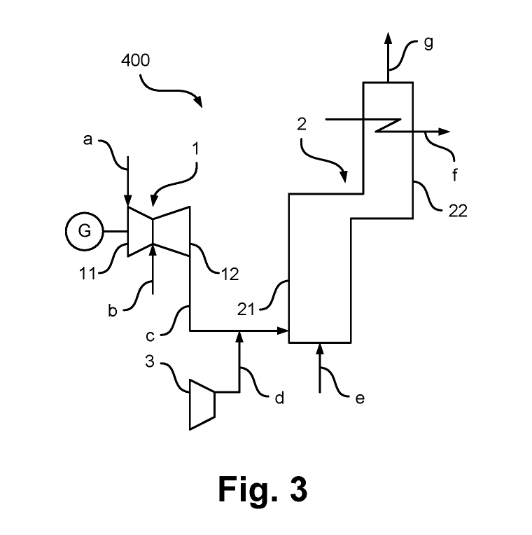

FIG. 3 is a simplified schematic representation of a combined installation with a gas turbine 1 and a fired reactor 2 according to the prior art, generally designated 400. The basic idea in the provision of such an installation 400 is to use the sensible heat of a combustion exhaust gas c from a gas turbine 1 similarly to a gas and steam power station, for example a gas and steam power station 300, as shown in FIG. 1, in a corresponding fired reactor 2. This makes use of the above mentioned fact that the combustion exhaust gas c still has a substantial oxygen content as a result of the significantly hyperstoichiometric combustion in the gas turbine 1. Additional combustion support gas d, for example air, is nevertheless supplied in the embodiment shown, for example fed into the combustion exhaust gas c by means of a blower 3.

This additional supply serves to provide an additional regulating variable for regulating the combustion in the reactor 2.

However, a significant disadvantage of combined installations 400 of this kind, according to the prior art, is that there is a significant reduction in the radiation zone efficiency of the fired reactor 2 in the radiation zone 21. By comparison with a self-sustaining reactor 2 as shown in FIG. 2, for example, the radiation zone efficiency decreases, for example, from about 0.42 to about 0.37. This can particularly be put down to the fact that, although the combustion exhaust gas c has a comparatively high temperature of for example about 600° C., its oxygen content of for example about 14% is nevertheless significantly below that of the combustion support gases such as combustion air which are typically used. Nor can this be compensated by the supply of additional combustion air d or a corresponding combustion support gas (at least not without exceptionally expensive oxygen enrichment). If air containing approximately 21% oxygen is used as the combustion support gas d in a reactor 2 operating in self-sustaining manner, as illustrated in FIG. 2, adiabatic combustion temperatures of about 2,000° C. can still be achieved by combustion in the radiation zone 21 of the reactor 2. By contrast, in an installation 400 as shown in FIG. 3, the adiabatic combustion temperature in the radiation zone 21 is limited to about 1,750° C. because of the circumstances described above. This is directly reflected in the poorer radiation zone efficiency stated.

Viewed in terms of energy balance, a considerable proportion of the chemical energy contained in the combustion support gas a is removed in the gas turbine 1 and is therefore no longer available thereafter in the combustion exhaust gas c.

Example data will now be provided for a gas turbine upstream of one or more reactors for steam cracking operating at an output of 1,000 MW with a maximised gas turbine power, i.e. minimum use of additional combustion support gas d for regulation. If about 1,132,000 Nm³/h of combustion air, for example, are used as the combustion support gas a in an installation 400 of this kind and if an underfiring power of about 340 MW is used in the form of the fuel b, about 118 MW of electrical power can be obtained at the efficiency levels explained previously in the generator G coupled to the gas turbine 1. Approximately 224 MW passes as sensible heat into the combustion exhaust gas c. Losses of about 5 MW are sustained, particularly at the generator G and subsidiary equipment and at the oil cooler of the gas turbine 1. The combustion exhaust gas c is formed in a quantity of about 1,170,000 Nm³/h.

In the embodiment shown, for example about 189,000 Nm³/h of combustion air are used as combustion support gas d. The underfiring power in the form of the fuel e is about 922 MW. The total heating power in the form of the fuels b and e used thus amounts to about 1,270 MW, the heating power available in the radiation zone 21 from the sensible heat of the combustion exhaust gas c and from the underfiring power in the form of the fuel e is about 1,147 MW. Of these, about 650 MW are recovered in the form of the pressurised steam fin an amount of about 756 t/h, about 69 MW go over into the cooled flue gas g which, as before, is removed at about 128° C. The quantity of the cooled flue gas g is thus about 1,457,000 Nm³/h, as compared to the above-mentioned approximately 1,172,000 Nm³/h in a self-sustaining reactor 2 as shown in FIG. 2. Here, too, 428 MW are discharged as chemical bonding energy and sensible heat in the tube-side process gas, as the same amount of reaction product is to be produced here as in the reactor 2 according to FIG. 2.

As already mentioned, the radiation zone efficiency in the radiation zone 21 is reduced to about 0.37. The efficiency level of the steam generation (from pressurised steam f) is about 0.51, the overall thermal efficiency is about 0.94. (Some of the heat which goes into the process gas in the radiation zone 21 is used for steam generation. Therefore, the two efficiency levels must not be added together or do not have to supplement one another to give the thermal efficiency specified. The quantity of heat and chemical bonding energy discharged with the process gas are absent from the total energy balance. However, these values are also identical in all the reactors 2 shown in the appended figures.)

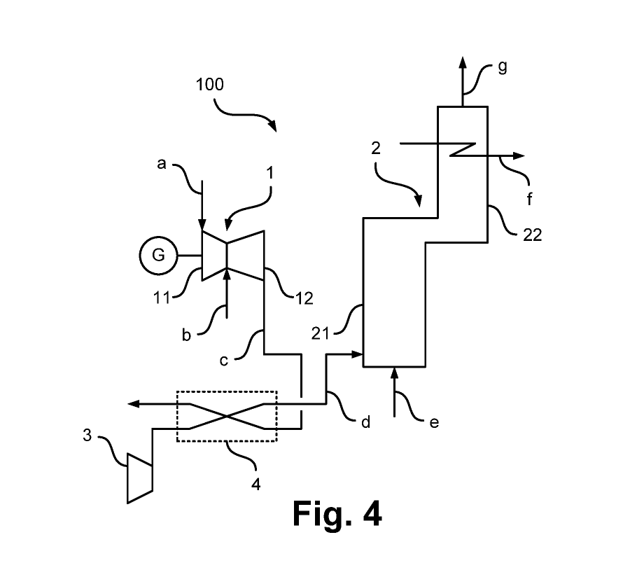

FIG. 4 shows a combined apparatus having a gas turbine 1 and a fired reactor 2 according to one embodiment of the invention, in a simplified schematic representation, the apparatus being generally designated 100.

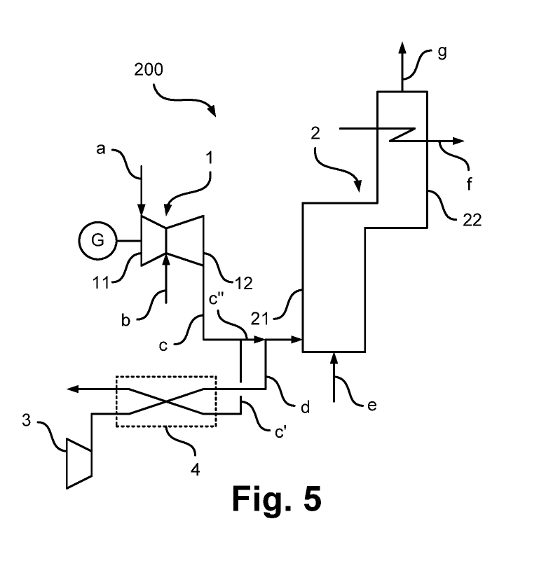

A central aspect of the present invention, as previously mentioned several times, is the use of a pre-heating unit 4 by means of which combustion support gas b fed in the reactor 2 is pre-heated. In the embodiment shown in FIG. 4, all of the combustion exhaust gas c from the gas turbine 1 is passed through the pre-heating unit 4, but it is also possible to use only a proportion of the combustion exhaust gas c. The latter is shown in the appended FIG. 5. By means of the pre-heating unit 4, which may for example comprise one or more suitably configured heat exchangers, sensible heat of the combustion exhaust gas 4 can be transferred to the combustion support gas d.

This has the particular advantage that a combustion support gas d such as combustion air which still has a high oxygen content can be fed into the reactor 2 but at the same time can be heated with the sensible heat of the combustion exhaust gas c. As has surprisingly been found, this substantially increases the radiation zone efficiency in the radiation zone 21 of the reactor 2, not only compared with reactors in corresponding coupled installations 400 as shown in FIG. 3 but also compared with self-sustaining reactors 2 as shown in FIG. 2. As a rule of thumb, 10° C. of pre-heating results in an increase of 0.2% in the radiation zone efficiency.

In the apparatus 100 shown in FIG. 4, a radiation zone efficiency level of about 0.47 is obtained in the radiation zone. When about 383,000 Nm³/h of combustion air are used as the combustion support gas a and an underfiring power of about 108 MW is used in the form of the stream b, an electrical power of about 40 MW can be obtained with the gas turbine 1 or the corresponding generator G. The combustion exhaust gas c is at about 656° C. (this is a value given by way of example, typical values being 550-700 CC), corresponding to a sensible heat of about 76 MW. Downstream of the pre-heating unit 4 the temperature of the combustion exhaust gas c is then still about 105° C., corresponding to a sensible heat of about 10 MW.

The temperature of the cooled combustion exhaust gas (flue gas temperature) is typically determined by the so-called “sulphur dew point”. At this temperature, aqueous sulphuric acid condenses, causing serious corrosion. The sulphur dew point is significantly lower at a lambda value of 3, (as in the flue gas of a gas turbine) than at a lambda value of 1.1 (in a steam cracking reactor), since proportionately a smaller quantity of (typically sulphur-containing) fuel or combustion product is present. The example values for a typical heating gas are 105° C. on the one hand and 128° C. on the other hand.

The quantity of combustion exhaust gas c is roughly 395,000 Nm³/h. If, in addition to this, about 879,000 Nm³/h of combustion air is provided as combustion support gas in the form of the stream d at about 28° C., for example, and this is heated in the pre-heating unit 4 to about 286° C., corresponding to a sensible heat of about 66 MW, and if the underfiring power in the form of the fuel e is about 824 MW, the total heating power available is about 942 MW and the heating power available in the reactor 2 is about 890 MW. Of these approximately 890 MW, a residue of about 462 MW is produced at the radiation zone efficiency of about 0.47, of which about 408 MW are obtained in the form of the pressurised steam f at about 475 t/h and about 54 MW are obtained in the form of the cooled flue gas g at about 128° C. or ab out 966,000 Nm³/h.

In a corresponding apparatus 100, the combustion support gas d may also be pre-heated to significantly higher temperatures, as illustrated, albeit only in the Tables (see below).

FIG. 5 shows a combined apparatus having a gas turbine 1 and a fired reactor 2 according to another embodiment of the invention, in simplified schematic representation, the apparatus being generally designated 200. The apparatus 200 differs from the apparatus 100 shown in FIG. 4 in that only part of the stream of combustion exhaust gas c is passed through the pre-heating unit 4. This partial stream is designated c′ in the apparatus 200. Another partial stream, here designated c″, is combined with the combustion support gas d. This results in particularly flexible operation of the installation 200, or the operating conditions of the reactor 2, as explained, can be approximated to those of a self-sustaining reactor 2 as shown in FIG. 2.

A corresponding embodiment of the invention according to FIG. 5 or installation 200 may comprise, in particular, providing the partial streams c′ and c″ in adjustable quantities, so as to enable adaptation to the respective heat supply in the combustion exhaust gas c and/or a heat requirement in the reactor 2. Once again, examples of characteristic values for an installation 200 are provided below.

If combustion air is provided in the installation 200 as combustion support gas a in an amount of about 1,035,000 Nm³/h and if an underfiring power in the form of fuel d of about 318 MW is used, an electrical power of about 107.8 MW can be generated in the gas turbine 1 at an efficiency level of about 0.34. The electrical efficiency in corresponding installations is somewhat lower than for gas turbines in a straightforward power station (cf. the explanations relating to FIG. 1: efficiency level therein 0.36), as additionally a pressure loss through the reactor 2 has to be overcome.

In the combustion exhaust gas c, overall, a sensible heat remains, corresponding to about 211 MW. If a partial stream c′ corresponding to a quantity of heat of about 77 MW is provided, a sensible heat corresponding to about 67 MW can be transferred by means of this partial stream c′ to combustion air, which in this case is used as combustion support gas d, in the preheating unit 4. Downstream of the preheating unit 4, about 10 MW of sensible heat remain in the stream c′, which is provided in an amount of about 391,000 Nm³/h per hour, corresponding to a temperature reduction from about 656° C. to 105° C. (see the explanations regarding FIG. 4 on the subject of sulphur dew point).

As already mentioned, combustion air, for example, is provided by means of the blower 3 as combustion support the gas d and is at a temperature of about 28° C. (the ambient temperature, by way of example). The quantity of combustion air is, for example, about 397,000 Nm³/h. In the preheating unit 4 the combustion air is heated to about 627° C., corresponding to the approximately 67 MW from the stream c′. The partial stream c″ of the combustion exhaust gas c is provided in a quantity of about 679,000 Nm³/h, corresponding to a sensible heat of about 134 MW. In addition, a fuel e corresponding to about 799 MW is supplied to the reactor 2.

Overall, therefore, a heating power of about 1,000 MW is available in the reactor 2 and overall a heating power of about 1,118 MW is available in the installation 200. By suitable adjustment of the streams c′ and c″, a radiation zone efficiency of about 0.42 can be achieved in the radiation zone 21 of the reactor 2, corresponding precisely to that of a self-sustaining reactor 2 as shown in FIG. 2. 512 MW remain in the pressurised steam f, which is provided at about 595 t/h, and about 60 MW remain in the cooled combustion exhaust gas c, of which about 1,172,000 Nm³/h are provided at 128° C.

In Tables 1 to 5 that follow, the flow quantities and energy contents previously mentioned with regard to FIGS. 1 to 5 are shown once again, wherein for FIG. 4 or the installation 100 illustrated therein, Tables 4A and 4B show two operational cases, namely preheating of the combustion support gas d to 286° C. (where conventional process control may take place in the reactor 2; see above) and to 498° C. (where further process changes need to be made to the reactor 2 such as steam generation with only partial superheating or an external/indirect preheating of the feed). In every case, air is used as the combustion support gas a or d and (residual) gas is used as the fuel. The values specified are to be understood as approximate values, disregarding any rounding-up errors. The heating power of the combustion exhaust gas c and of the cooled combustion exhaust gas g corresponds to the sensible heat, the heating power of the pressurised steam f corresponds to the sum of the sensible heat and the evaporation enthalpy.

TABLE 1
Gas and Steam Power Station 300 (FIG. 1)

| | | |
|---|---|---|
| Current production gas turbine 1 | 64 | MW |
| Flow quantity combustion support gas a | 619,000 | Nm³/h |
| Firing power fuel b | 180 | MW |
| Heat loss | 3 | MW |
| Heating power of combustion exhaust gas c | 112 | MW |
| Flow quantity of combustion exhaust gas c | 640,000 | Nm³/h |
| Heating power of pressurised steam f | 112 | MW |
| Current production of decompression turbine 6 | 36 | MW |
| Power discharged into cooler 7 | 44 | MW |
| Heating power of cooled exhaust gas g | 33 | MW |
| Flow quantity of cooled exhaust gas g | 640,000 | Nm³/h |
| Temperature of cooled exhaust gas g | 128° | C. |
| Overall electrical efficiency level | 0.56 | |

TABLE 2
Self sustaining fired reactor 2 (FIG. 2)

| | | |
|---|---|---|
| Flow quantity of combustion support gas d | 1,067,000 | Nm³/h |
| Firing power of fuel e | 1,000 | MW |
| Radiation zone efficiency of radiation zone 21 | 0.42 | |
| Heating power of pressurised steam f | 512 | MW |
| Flow quantity of pressurised steam f | 595 | t/h |
| Heating power of cooled exhaust gas g | 60 | MW |
| Flow quantity of cooled exhaust gas g | 1,172,000 | Nm³/h |
| Temperature of cooled exhaust gas g | 128° | C. |

TABLE 3
Combined installation 400 (FIG. 3)

| | | |
|---|---|---|
| Current production of gas turbine 1 | 118 | MW |
| Flow quantity of combustion support gas a | 1,132,000 | Nm³/h |
| Firing power of fuel b | 348 | MW |
| Heating power of exhaust gas c | 224 | MW |
| Flow quantity of exhaust gas c | 1,170,000 | Nm³/h |
| Flow quantity of combustion support gas d | 189,000 | Nm³/h |
| Firing power of fuel e | 922 | MW |
| Radiation zone efficiency of radiation zone 21 | 0.36 | |
| Heating power of pressurised steam f | 650 | MW |
| Flow quantity of pressurised steam f | 756 | t/h |
| Heating power of cooled exhaust gas g | 69 | MW |
| Flow quantity of cooled exhaust gas g | 1,457,000 | Nm³/h |
| Temperature of cooled exhaust gas g | 128° | C. |

TABLE 4A
Apparatus100, Embodiment of the invention (FIG. 4), 286° C.

| | | |
|---|---|---|
| Current production of gas turbine1 | 40 | MW |
| Flow quantity of combustion support gas a | 383,000 | Nm³/h |
| Firing power of fuel b | 118 | MW |
| Heating power of exhaust gas c | 76 | MW |
| Flow quantity of exhaust gas c | 395,000 | Nm³/h |
| Temperature of exhaust gas c | 656° | C. |
| Transferred in preheating device 4 | 66 | MW |
| Heating of combustion support gas d from | 28° | C. |
| Heating of combustion support gas d to | 286° | C. |
| Flow quantity of combustion support gas d | 879,000 | Nm³/h |
| Heating power of fuel e | 824 | MW |
| Radiation zone efficiency of radiation zone 21 | 0.47 | |
| Heating power of pressurised steam f | 408 | MW |
| Flow quantity of pressurised steam f | 475 | t/h |
| Heating power of cooled combustion exhaust gas g | 69 | MW |
| Flow quantity of cooled combustion exhaust gas g | 966,000 | Nm³/h |
| Temperature of cooled combustion exhaust gas g | 128° | C. |

TABLE 4B
Apparatus 100, Embodiment of the invention (FIG. 4), 498° C.

| | | |
|---|---|---|
| Current production of gas turbine 1 | 60 | MW |
| Flow quantity of combustion support gas a | 580,000 | Nm³/h |
| Heating power of exhaust gas c | 115 | MW |
| Flow quantity of exhaust gas c | 599,000 | Nm³/h |
| Temperature of exhaust gas c | 656° | C. |
| Transfer in preheating device 4 | 100 | MW |
| Heating of combustion support gas d from | 28° | C. |
| Heating of combustion support gas d to | 498° | C. |
| Flow quantity of combustion support gas d | 765,000 | Nm³/h |
| Firing power of fuel e | 717 | MW |
| Radiation zone efficiency of radiation zone 21 | 0.51 | |
| Heating power of pressurised steam f | 335 | MW |
| Flow quantity of pressurised steam f | 390 | t/h |
| Heating power of cooled exhaust gas g | 50 | MW |
| Temperature of cooled exhaust gas g | 128° | C. |

TABLE 5
Apparatus 200, Embodiment of the invention (FIG. 5)

| | | |
|---|---|---|
| Current production of gas turbine 1 | 108 | MW |
| Flow quantity of combustion support gas a | 1,035,000 | Nm³/h |
| Firing power of fuel b | 318 | MW |
| Heating power of combustion exhaust gas c | 211 | MW |
| Flow quantity of combustion exhaust gas c | 765,000 | Nm³/h |
| Temperature of combustion exhaust gas c | 656° | C. |
| Energy transferred in preheating device 4 | 77 | MW |
| Heating of combustion support gas d from | 28° | C. |
| Heating of combustion support gas d to | 627° | C. |
| Firing power of fuel e | 799 | MW |
| Radiation zone efficiency of radiation zone 21 | 0.42 | |
| Heating power of pressurised steam f | 512 | MW |
| Flow quantity of pressurised steam f | 595 | t/h |
| Heating power of cooled exhaust gas g | 60 | MW |
| Flow quantity of cooled exhaust gas g | 1,172,000 | Nm³/h |
| Temperature of cooled exhaust gas g | 128° | C. |

The following tables 6A to 6C represent comparable processes for illustrating the advantages of the present invention over one another. In all the Tables the same amount of current (line “current production” in the Table) should be produced in the installation as a whole and an equal amount of a reaction product (in this case the hydrocarbon ethylene, line “ethylene production” in the Table) is to be produced in a reactor 2. Reference is explicitly made to the above Figures and particularly to the introductory paragraphs of the description of the drawings.

Moreover it is assumed that current is produced with the pressurised steam f. This serves predominantly for improved comparability of the processes in terms of their electrical efficiency and the “efficiency” in the sense of the above definitions. The line “current production” in the Table or the values given therein thus also encompass the current generated from the pressurised steam f, assuming a typical electrical efficiency of 0.24 for pressurised steam f at 520° C. and 120 bar.

Column 1 of the Tables (“Reactor, mains current”) contains values for current supplied by a mains supply and for a reactor 2 operated in self-sustaining manner according to FIG. 2. For the generation of the current supplied from the mains supply, an efficiency of 0.33 is assumed. This corresponds to a typical evaluation number for current from conventional mains supplies (i.e. the average electrical efficiency over a network of power stations from a supplier, comprising old and new power stations of all kinds, i.e. pure (coal) steam power stations and gas and steam power stations and also including all line loss). Thus the current is supplied from the mains according to column 1 if it is not generated from the pressurised steam f.

The firing power corresponding to an efficiency level of 0.33 which would be required to generate this proportion of current supplied from the mains is included in the total heating power required (the line “total heating power” in the Table) which is required in addition to the heating power for the reactor 2 to make the processes comparable. This heating power is additionally given as heating power standardised to the heating power specified in column 2 of the Table (the line “heating power %” in the Table).

Column 2 of the table (“reactor, gas and steam power station”) gives values for a combination of a separate gas and steam power station, e.g. according to FIG. 1, and a self-sustaining reactor 2 according to FIG. 2, e.g. as already specified in column 1 of the Table. The heating power required for current production in the gas and steam power station depends on the overall electrical efficiency of 0.56 assumed here (see the remarks regarding FIG. 1) and is included in the total heating power stated in the above-mentioned lines of the Table (in addition to the heating power for the reactor 2).

A look at columns 1 and 2 together shows that, for the same production of electrical power (the line “Current production” in the Table), which comprises current taken from the mains (column 1 of the Table) or generated in the gas and steam power station (column 2 of the Table), and the same production quantity of the reaction product ethylene, a reduction in the heating power required can be achieved straight away on the basis of the different electrical efficiencies (0.56 overall electrical efficiency in current generation in a gas and steam power station, see the comments on FIG. 1; 0.33 for typical mains current, see above) according to column 2 of the Table.

The specific energy consumption of the process based on the same quantity of the reaction product ethylene (the line “specific energy consumption” in the Table) is reduced accordingly, so that the “energy efficiency” of the process in the sense explained hereinbefore is increased accordingly. The efficiency levels relating to the reactor 2 do not change, since the reactor 2 continues to operate self sufficiently.

In Table 6A the values of columns 1 and 2 of the Table explained above are compared in column 3 (“combined installation 400 according to FIG. 3”) with values relating to a combined installation 400 according to FIG. 3, at an assumed total current production of 152 MW. Of these 152 MW, according to column 3 of the Table 118 MW are generated by the gas turbine 1 and 34 MW are assumed to be generated from the pressurised steam f at a corresponding (low) efficiency (this simplification is for comparison purposes, even if the utilisation of the pressurised steam fin practice is typically the direct use of shaft power to drive compressors or pumps—which in any case corresponds to the electrical power, apart from a marginal loss at the generator of typically about 1%).

Because of the radiation zone efficiency which has deteriorated from about 0.42 to about 0.37, in this case an increased heating power has to be used in the reactor 2 for the same amount of reaction products. For this reason the heating power according to column 3 of the Table is 1,270 MW in all, which admittedly represents a significant improvement over column 1 of the Table but only a marginal improvement of 0.1% over column 2 of the Table.

TABLE 6A

| | | | Reactor, | Combined |
|---|---|---|---|---|
| | | | Gas and | installation |
| | | Reactor, | Steam | 400 according |
| | | Mains | Power | to |
| | | Current | Station | FIG. 3 |
| Parameter | Unit | 1 | 2 | 3 |
| Ethylene production | t/h | 176 | 176 | 176 |
| Current production | MW | 152 | 152 | 152 |
| Specific energy | Gcal/t | 6.85 | 5.93 | 5.93 |
| consumption | | | | |
| Specific energy | | 115.4% | 100.0% | 99.9% |
| consumption % | | | | |
| Heating power total | MW | 1,459 | 1,272 | 1,270 |
| Heating power % | | 114.7% | 100.0% | 99.9% |

The fuel consumption for generating electrical energy is determined in the two benchmark cases according to columns 1 and 2 (i.e. with separate and self-sustaining production of current and reaction products) for the electric power which is possible in coupled production. (Thus the benchmark case is “adapted”, with the knowledge that gas and steam power stations with less than 80 MW electric power are scarcely likely to be set up as independent power stations. This also applies to the following Tables. Heating power and specific energy consumption correlate with one another, as the heating requirement makes up significantly more than half the total energy consumption.

In Table 6B the values of columns 1 and 2 of the Table which have already been described in connection with those in Table 6A and which have the same meaning as in Table 6A are compared in column 3 (“combined installation according to FIG. 5”) with values relating to a combined installation 200 according to FIG. 3 with an assumed current production of 108 MW. This is the current production of the gas turbine 1, as the steam production in the reactor 2 is the same as the steam production in a self-sustaining reactor.

Thanks to the distribution of the combustion exhaust gas c over partial streams c′ and c″ and the partial use only for preheating in the preheating device 4 and the partial feeding into the reactor 2, in a combined installation 200 according to FIG. 5 the radiation zone efficiency can be kept constant here compared with columns 1 and 2 of the Table, namely to the value of about 0.42 mentioned above. With the specific combination of parameters given, the heating power required is therefore significantly reduced, i.e. by about 6% compared with column 2 of the Table and by about 17% compared with column 1 of the Table. The specific energy consumption, based on the same quantity of the reaction product ethylene, is also significantly reduced accordingly. At the same time, as already mentioned, a corresponding reactor 2 can be continued to be operated under conventional conditions.

TABLE 6B

| | | | Reactor, | Combined |
|---|---|---|---|---|
| | | | Gas and | Installation 200 |
| | | Reactor, | Steam | according |
| | | Mains | Power | to |
| | | Current | Station | FIG. 5 |
| Parameter | Unit | 1 | 2 | 3 |
| Ethylene production | t/h | 176 | 176 | 176 |
| Current production | MW | 108 | 108 | 108 |
| Specific energy | Gcal/t | 6.58 | 5.93 | 5.56 |
| consumption | | | | |
| Specific energy | | 111.0% | 100.0% | 93.8% |
| consumption % | | | | |
| Heating power total | MW | 1,327 | 1,193 | 1,118 |
| Heating power % | | 111.2% | 100.0% | 93.7% |

In Table 6C, the values of columns 1 and 2 of the Table have been compared in column 3 (“Combined Apparatus according to FIG. 4, 498° C.”) with values relating to a combined installation 100 according to FIG. 4 with preheating of the combustion support gas d to 498° C., i.e. according to Table 4B, and with an assumed current production of 60 MW by the gas turbine. Of these 60 MW, 43 MW have to be taken off (the line “reduced steam production as electric power” in the Table) as less steam is produced and the corresponding shortfall of shaft power is simply compensated by electrical power. Losses of typically about 3% on an electric engine used are ignored in the interests of simplicity.

Because of the radiation zone efficiency which is once again dramatically increased, namely to a value of about 0.53, there is another significant reduction in the heating power while current production is identical compared with columns 1 and 2 of the Table.

TABLE 6C

| | | | | Combined |
|---|---|---|---|---|
| | | Reactor, | Reactor, Gas | installation 100 |
| | | Mains | and Steam | according to |
| | | Current | Power Station | FIG. 4, 498° C. |
| Parameter | Unit | 1 | 2 | 3 |
| Ethylene | t/h | 176 | 176 | 176 |
| Production | | | | |
| Current Production | MW | 60 | 60 | 60 |
| Specific Energy | Gcal/t | 6.03 | 5.93 | 5.27 |
| Consumption | | | | |
| Specific Energy | | 101.7% | 100.0% | 88.8% |
| Consumption % | | | | |
| Total Heating | MW | 1.052 | 1.013 | 896 |
| Power | | | | |
| Heating Power % | | 102.0% | 100.0% | 86.90% |
| Reduced Steam | MW | 0 | 0 | 43 |
| Production as | | | | |
| Electric Power | | | | |
| Net Steam | MW | 17 | 17 | 17 |
| Production | | | | |

A combined study of the above Tables will show, in particular, that the partial power generation from a (high grade) fuel, for example a residual gas from a corresponding process carried out using the reactor 2, achieves an increase in efficiency of about 11% compared with the generation of current using a typical power station mix or by taking from the mains, regardless of whether a gas and steam power station according to FIG. 1 or a known combination in the form of an installation 400 according to FIG. 3 is used. The pre-requisite here is, in particular, sufficient availability of a suitable residual gas, for example. Moreover, it will be seen that the known combination in the form of an installation 400 according to FIG. 3 has no or only slight efficiency advantages over the separate generation of current and reaction products, i.e. in the examples shown above, the same heating power and hence a comparable fuel supply are required.

The installation 200 proposed according to one embodiment of the invention has, by comparison, a roughly 6% higher efficiency compared with separate current production by means of a gas and steam power station and the separate manufacture of the reaction products in a self-sustaining reactor 2. Compared with a typical power station mix or the taking of current from the mains and the separate generation of the reaction products in a self-sustaining reactor 2, an increase in efficiency of about 11% is observed.

The installation 200 generates 92% current per additional unit of firing power used (i.e. per 1 MW heating power used in addition to the heating power required for the self-sustaining reactor operation, 0.92 MW of current are generated). This corresponds to virtually double the electrical efficiency of a gas and steam power station or three times the electrical efficiency of a power station mix or the taking of current from the mains.

The proposed installation 100 requires less heating power than the self-sustaining reactor and also produces additional current. The installation 100 thus has a roughly 11% higher efficiency compared with the separate generation of current by means of a gas and steam power station and the separate manufacture of the reaction products in a self-sustaining reactor 2.

1. Process for the combined generation of mechanical power and manufacture of reaction products, wherein in order to generate the mechanical power at least one internal combustion engine (1) is fired up, thereby producing a combustion exhaust gas (c), and wherein in order to produce the hydrocarbons at least one reactor (2) is heated using a fuel (e) and a combustion support gas (d), characterised in that at least a proportion of the combustion support gas (d) is heated by indirect heat exchange with at least a proportion of the combustion exhaust gas (c) from the internal combustion engine (1).

2. Process according to claim 1, wherein the mechanical power is converted at least partially into electrical power by means of at least one generator (G) and/or is used to drive at least one shaft.

3. Process according to claim 1 or 2, wherein the at least one reactor (2) is configured as a tube reactor in which, in a radiation zone, reaction tubes are heated from outside by burners in which the fuel is being combusted.

4. Process according to claim 3, wherein, in order to produce the hydrocarbons, a feed is passed, by means of pressurised steam, through the reaction tubes of the reactor (2) configured as a tube reactor.

5. Process according to claim 3 or 4, wherein a catalyst is provided in the reaction tubes of the reactor (2) configured as a tube reactor.

6. Process according to one of the preceding claims, wherein the fuel (e) is formed at least partly from a gas mixture which is separated from a product stream from the at least one reactor (2).

7. Process according to one of the preceding claims, wherein at least one region of the at least one reactor (2) is heated to a temperature level of 1,500 to 2,500° C., particularly to a temperature level of 1,500 to 2,500° C. in the at least one region of the at least one reactor (2) by heating using the fuel (e) and the combustion support gas (d).

8. Process according to one of the preceding claims, wherein the at least one internal combustion engine (1) comprises at least one gas turbine.

9. Process according to one of the preceding claims, wherein the combustion exhaust gas (c) from the internal combustion engine (1) is provided at a temperature level of 500 to 1,000° C., particularly at a temperature level of 600 to 700° C. or at a temperature level of 500 to 650° C.

10. Process according to one of the preceding claims, wherein a proportion of the combustion exhaust gas (c) from the internal combustion engine (1) is used to heat the combustion support gas (d) by indirect heat exchange and a proportion of the combustion exhaust gas (c) from the internal combustion engine (1) is combined with the combustion support gas (d) and is supplied together therewith to the at least one reactor (2).

11. Process according to one of claims 1 to 9, wherein the combustion exhaust gas (c) from the internal combustion engine (1) is used in its entirety to heat the combustion support gas (d) by indirect heat exchange and is not supplied to the at least one reactor (2).

12. Process according to one of the preceding claims, wherein natural gas and/or a methane-containing gas mixture, particularly a gas mixture formed according to claim 6, which contains hydrogen, methane and carbon monoxide, is used as the fuel (e) and/or air is used as the combustion support gas (d).

13. Process according to one of the preceding claims, wherein pressurised steam (f) is produced from the waste heat from the at least one reactor (2) and is used to drive at least one shaft, particularly of a generator (G).

14. Installation (100, 200) for the combined generation of mechanical power and production of reaction products, which comprises, for the generation of the mechanical power, at least one internal combustion engine (1) which can be fired up, thereby producing a combustion exhaust gas (c), and which comprises, for the production of the hydrocarbons, at least one reactor (2) which can be heated using a fuel (e) and a combustion support gas (d), characterised in that means are provided which are arranged so as to heat at least a proportion of the combustion support gas (d) by indirect heat exchange with at least a proportion of the combustion exhaust gas (c) from the internal combustion engine (1).

15. Installation (100, 200) according to claim 14, which is arranged so as to carry out a process according to one of claims 1 to 13.
